(19) United States
(12) Patent Application Publication
(10) Pub. No.: US 2007/0179582 A1
(43) Pub. Date: Aug. 2, 2007

(54) Polymer reinforced coil conductor for torque transmission

(76) Inventors: Mark T. Marshall, Forest Lake, MN (US); Henry D. Shroder, Saint Louis Park, MN (US)

Correspondence Address: MEDTRONIC, INC., 710 MEDTRONIC PARKWAY NE, MINNEAPOLIS, MN, 55432-9924, US

(21) Appl. No.: 11/343,884

(22) Filed: Jan. 31, 2006

(51) Int. Cl. A61N 1/05

(52) U.S. Cl. 607/119

(57) ABSTRACT

A coil conductor for connecting an electrode near a distal end of a medical electrical lead with an implantable medical device (IMD) connected with a proximal end of the medical electrical lead includes a multi-filar coil and a torque enhancing sheathing. The multi-filar coil comprises a co-radially wound, multi-filar coil that has an inductance of approximately 1.5 μH or greater. The sheathing is extruded over to enhance the torque transmitting properties of the coil conductor.

### CROSS REFERENCE TO RELATED APPLICATION(S)

The following co-pending application is filed on the same day as this application: “MEDICAL ELECTRICAL LEAD HAVING IMPROVED INDUCTANCE” by M. T. Marshall and K. R. Seifert (attorney docket number P20787), and is incorporated herein by reference.

### BACKGROUND OF THE INVENTION

The present invention relates generally to implantable medical device (IMD) leads for delivering electrodes to various places in a human body, such as the heart. In particular, the present invention relates to leads having a torque coil for securing lead fixation devices that are also compatible with radio frequency (RF) fields generated by magnetic resonance imaging (MRI).

Typical leads for use with an IMD, such as an implantable cardioverter defibrillation (ICD) device, deliver multiple conductors to the heart for performing pacing, cardioverting, defibrillating, sensing and monitoring functions. One of these conductors comprises a multi-filar coil that is connected with a tip electrode and, along with the IMD, performs the pacing and sensing functions. In some embodiments, the tip electrode includes a fixation device, such as a helix or corkscrew, which connects the tip electrode and coil conductor with heart tissue. In order to secure the fixation device to the tissue, it is necessary to extend the fixation device from the lead body and then to screw it into the heart tissue, which is typically accomplished by applying a rotational force to the fixation device. During implanting of the lead, the coil conductor is rotated at its proximal end to extend and secure the fixation helix at its distal end. Thus, it is necessary for the coil conductor to transmit the applied torque along its length from the proximal end to the distal end. Typically, coil conductors having as many as five filars with a large pitch have been used in order to transmit the necessary torque to the fixation device. These multi-filar, high pitch coil conductors, however, have very low inductance. During magnetic resonance imaging, it is necessary to expose the patient and the IMD to a radio-frequency field, which is used to generate the MRI image. Generally, it is desirable for a lead conductor to have increased inductance in order to minimize excitation and heating effects from RF fields generated during magnetic resonance imaging.

### BRIEF SUMMARY OF THE INVENTION

The present invention comprises a coil conductor with a torque enhancing sheathing for connecting an electrode near a distal end of a medical electrical lead with an implantable medical device (IMD) connected with a proximal end of the medical electrical lead. The coil conductor comprises a co-radially wound, multi-filar coil that forms a circuit between the electrode and the IMD, and includes an inductance of approximately 1.5 μH or greater. The sheathing enhances the torque transmitting properties of the coil.

### BRIEF DESCRIPTION OF THE DRAWINGS

FIG. 1 shows a medical electrical lead of the present invention for use with an implantable cardioverter defibrillation (ICD) device.

FIG. 2A shows cross section 2-2 of FIG. 1 showing the conductors of the ICD lead.

FIG. 2B shows a partially cut away perspective view of cross section 2-2 of FIG. 1.

FIG. 3 shows cross section 3-3 of FIG. 2A.

### DETAILED DESCRIPTION

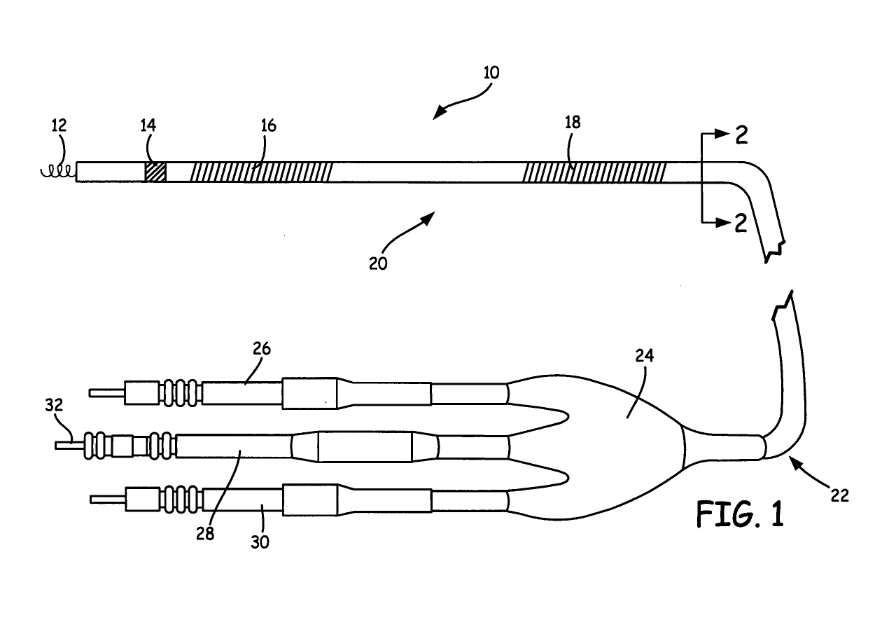

FIG. 1 shows implantable cardioverter defibrillation (ICD) lead 10 of the present invention. ICD lead 10 is used to deliver tip electrode 12, ring electrode 14, right ventricle (RV) defibrillation coil 16 and superior vena cava (SVC) defibrillation coil 18 to a heart for the purposes of providing cardio-therapy.

Tip electrode 12, ring electrode 14, RV coil 16 and SVC coil 18 are connected at distal end 20 of ICD lead 10 with various conductors that run to proximal end 22 of ICD lead 10, where the conductors are joined with connector assembly 24. Connector assembly 24 routes the individual conductors to connectors 26, 28 and 30 for connection with connector sockets of an implantable medical device (IMD).

Tip electrode 12 and ring electrode 14 are connected with connector 28 and with a conductor coil and a conductor cable, respectively, which are electrically isolated within lead 10. Tip electrode 12 and ring electrode 14 are used to sense cardiac signals and to deliver pacing pulses to the right ventricle of the heart in conjunction with the IMD. RV coil 16 is joined with connector 26, and SVC coil 18 is joined with connector 30 through conductor cables, which are electrically isolated from each other within in lead 10. RV coil 16 (which is placed in the right ventricle) and SVC coil 18 (which is placed in the superior vena cava) can be used as cathode and anode to deliver defibrillation shocks to the heart from the IMD, as a result of a tachycardia or fibrillation condition sensed in the heart by tip electrode 12 and ring electrode 14.

Typically, tip electrode 12 comprises a fixation helix, which is used to secure tip electrode 12 to tissue of the right ventricular apex of the heart. The fixation helix comprises a rigid coil with a sharpened tip that can penetrate into the tissue in order to anchor the position of tip electrode 12 within the heart. Once tip electrode 12 is properly positioned within the heart during implanting of lead 10, the fixation helix is rotated so that its tip will penetrate the heart tissue. (In some embodiments, the rotational force is also used to extend the fixation helix from within the body of lead 10.) The rotational force is transmitted to the fixation helix through a conductor coil for connecting tip electrode 12 with connector pin 32 of connector 28. Thus, the conductor coil must be capable of transmitting a rotational force applied to connector pin 32 to the fixation helix.

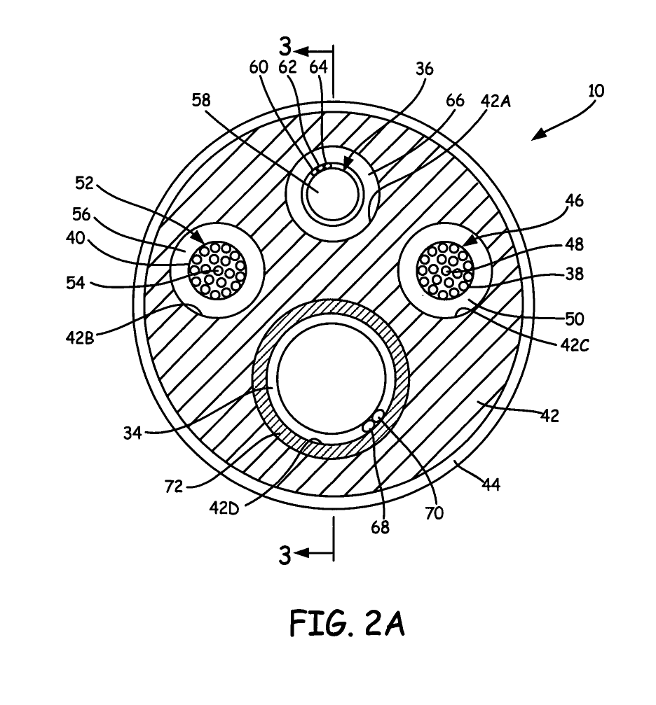

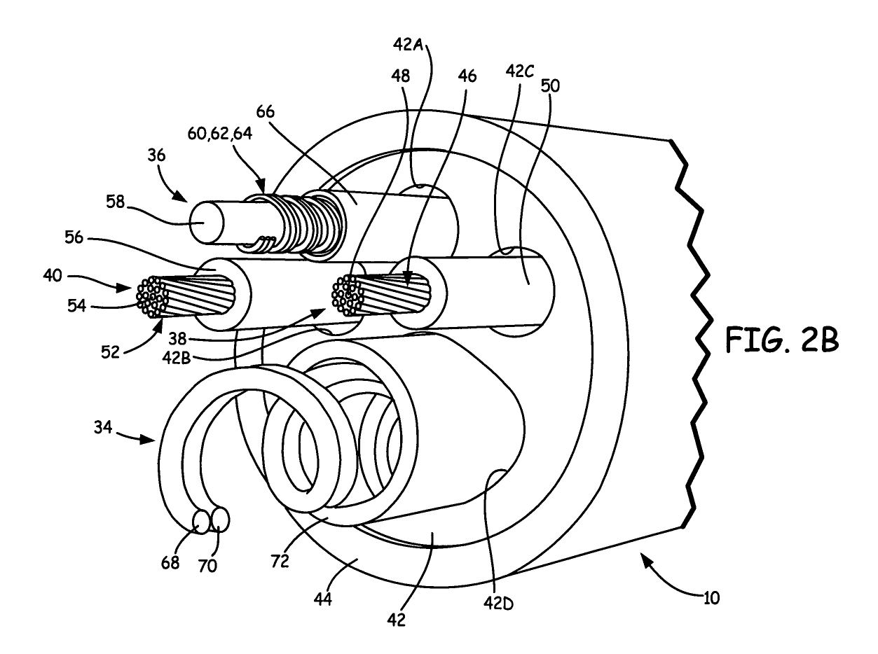

FIG. 2A shows cross section 2-2 of FIG. 1 showing the conductors of lead 10, including coil conductor 34, sense conductor 36, RV conductor 38 and SVC conductor 40. FIG. 2B shows a partially cut away perspective view of cross section 2-2 of FIG. 1, in which the features of lead 10 are illustrated. FIGS. 2A and 2B are discussed concurrently.

ICD lead 10 includes multi-lumen lead body 42, which includes four lumens 42A-42D for conveying each of the four conductors of lead 10. Lead body 42 is typically comprised of extruded silicone rubber, and is covered by sheathing 44 that protects the components of lead 10 from the environment of the body in which it is implanted. Sheathing 44 is also comprised of extruded silicone rubber or another bio-compatible material.

As discussed above, exposure of IMD leads to MRI can result in localized heating of electrodes due to excitation of conductors from RF fields used in obtaining MRI images. When an electrode with a small surface area is vibrated by a conductor, heat can build up in the electrode. High levels of vibration in an electrode are correlated with low inductance of the conductor to which it is connected. Conductors with high inductance are more resistant to excitation in RF fields, and are therefore more RF field compatible. For small electrodes, it is desirable to connect them with the IMD using conductors having a higher inductance.

Generally, it is desirable for conductors used in conjunction with tip electrodes to have a total inductance in the range of about 1.0 μH to about 5.0 μH, preferably greater than about 1.5 μH. A large inductance is necessary due to the relative small surface area of tip electrodes, typically about 2.5 mm² (˜0.003875 in²). For ring electrodes, which have surface areas in the range of about 34 mm² (˜0.0527 in²), the inductance of the conductor may be as low as approximately 0.5 μH, but is preferably higher.

The inductance of a conductor is determined by its geometric properties, particularly if it is wound into a coil or straight. Straight wires have an inductance that approaches zero, and are therefore generally undesirable for small electrodes of leads that have the possibility of exposure to MRI. A conductor that includes straight filars in addition to wound filars also has an inductance that approaches zero.

For coiled or wound conductors, several parameters are determinative of its inductance: the diameter of each wire conductor, the pitch of the coil (the distance between turns of the coil), the cross-sectional area occupied by the coil, and the number of filars comprising the coil. These parameters are constrained by the design requirements for each application in which the lead will be used. For example, a typical ICD lead must have an overall diameter less than approximately 6.6 French (˜0.0866″ or ˜0.2198 cm).

RV conductor 38 comprises a stranded cable conductor in which nineteen wire filars 46 are wrapped around central wire filar 48 inside sheathing 50. Similarly, SVC conductor 40 comprises a stranded cable conductor in which nineteen wire filars 52 are wrapped around central wire filar 54 inside sheathing 56. The inductance of straight, central filars 48 and 52 effectively reduces the inductance of conductors 38 and 40 to zero. However, because RV conductor 38 and SVC conductor 40 are connected with RV coil 16 and SVC coil 18, which have large enough surface areas, excitation heating is not a concern and neither is the inductance of conductors 38 and 40.

Conductor 36 is connected with ring electrode 14, which has a relatively small surface area and is thus susceptible to excitation heating. Therefore, the inductance of conductor 36 is increased to be RF field compatible utilizing an improved design, the details of which are described in the above referenced co-pending application by Marshall and Seifert. In short, the inductance of sense conductor 36 is improved by replacing the central, straight filar with non-conducting fiber strand 58. This eliminates the inductance of the straight wire filar, which dominates the inductance of the entirety of conductor 36. Replacing the nineteen wire filars are wire filars 60, 62 and 64, which are wound around core fiber 58 in a manner that increases the inductance of sense conductor 36. Conductor 36 is wrapped in sheathing 66, which acts as an insulator and as a protective barrier.

Turning to the present invention, coil conductor 34 is connected with tip electrode 12, which has a relatively small surface area. Therefore, it is important for coil conductor 34 to have a high enough inductance to be RF field compatible. The inductance of coil conductor 34 is important, but must be achieved while also maintaining the torque transmitting capabilities of coil conductor 34. Coil conductor 34 is comprised of co-radially wound filars 68 and 70, that are enveloped in compression sheathing 72.

In order to produce the torque transmitting capabilities necessary for securing a fixation helix with tissue, a typical torque coil consists of a five-filar coil wound with a very high pitch. Five-filar designs, with multiple small diameter wires, have been the preferred design for torque transmission because they have the advantage of staying within diameter and flexibility requirements necessary for medical electrical leads, as opposed to designs with fewer or thicker filars, which are larger and less flexible. Therefore, it has typically been the case to use multiple filars with a high pitch to obtain the necessary torque transmitting capabilities.

In order to increase the inductance of a torque coil, the pitch could be decreased, the coil diameter could be increased, or the number of filars could be reduced. However, the diameter cannot be increased due to size limitations of lead 10, and the number of coils cannot be reduced or the pitch decreased without sacrificing torque transmitting capabilities. Coil conductor 34 of the present invention resolves the competing interests between inductance and torque transmission by adding compression sheathing 72 to a high inductance coil conductor 34. Compression sheathing 72 enhances the torque transmission of coil conductor 34, without which coil conductor 34 may not be able to transmit sufficient torque to tip electrode 12.

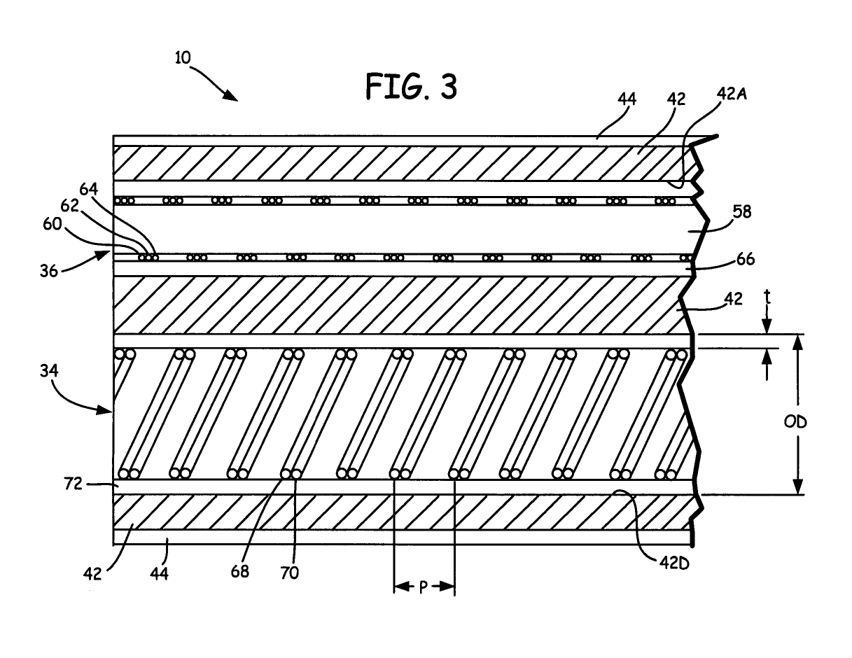

FIG. 3 shows cross-section 3-3 of FIG. 2A, illustrating a longitudinal cross-section of lead 10 and the winding of coil conductor 34. Lead 10 includes coil conductor 34 and conductor 36, which are interposed in multi-lumen lead body 42 and wrapped in sheathing 44.

Conductor 36 includes conductor filars 60, 62 and 64, which are wound around fiber core 58 and encased in sheathing 66. Conductor 36 is connected with ring electrode 14 at its distal end and with connector 28 at its proximal end, and is used in conjunction with coil conductor 34 to perform typical sensing and pacing operations.

Coil conductor 34 includes conductor filars 68 and 70, which are wrapped in compression sheathing 72, which also acts as an insulator and protective barrier. Coil conductor 36 is connected with tip electrode 12 at its distal end and with connector 28 at its proximal end and is used to deliver pacing stimuli to the heart.

As compared with previous designs, the number of filars of coil conductor 34 is reduced from the typical five to two: filars 68 and 70. Since only two filars are used in coil conductor 34, the pitch of coil conductor 34 is decreased such that the winding of filars 68 and 70 are denser than in previous designs. (In FIG. 3, the pitch is not shown to scale and is exaggerated for clarity.) The decreased pitch and reduced number of filars serve to increase the inductance of coil conductor 34 such that an RF field compatible inductance is reached, and can be modified for other designs and depending on the filar diameter. In one embodiment, filars 68 and 70 are comprised of a 0.0012″ (˜0.0305 mm) diameter cobalt based sheath, silver core wire such as MP35N® wire, and the pitch p of coil conductor 34 is 0.006″ (˜0.152 mm). The pitch p of coil conductor 34 can be reduced to the diameter of filars 68 and 70, depending on the torque enhancing characteristics of compression sheathing 72.

Other embodiments use 0.002″ (˜0.0508 mm) diameter wire with a 0.0159″ (˜0.4039 mm) core, or 0.003″ (˜0.0762 mm) diameter wire with a 0.0179″ (˜0.4547 mm) core. In other embodiments, similar wire materials can be used, such as tantalum sheathings, or silver or gold cores.

Compression sheathing 72 comprises a polymer jacket that is extruded over coil conductor 34. In one embodiment, compression sheathing 72 extends continuously from near the proximal end of coil conductor 34 to near the distal end of coil conductor 34. Compression sheathing 72 strengthens and reinforces the windings of coil conductor 34 by bonding to, and forming over filars 68 and 70 a rigid jacket. Thus, compression sheathing 72 restricts filars from expanding (“bird caging”) or contracting in the radial direction when under torque, yet does not unduly burden the longitudinal flexibility of coil conductor 34. Thus, in one embodiment compression sheathing 72 slightly constricts coil conductor 34, but not enough to increase the stiffness of coil conductor 34 so it interferes with insertion of lead 10. In another embodiment, compression sheathing 72 does not compress coil conductor 34 at all, but only prevents it from expanding. The thickness of compression sheathing 72 is determined by the diameter restrictions of coil conductor 34 and lead 10, and the desired torque transmission capabilities of coil conductor 34. Typically, the outer diameter, OD, of coil conductor 34, including any sheathing or insulation, is about 0.025″ (˜0.635 mm). In one embodiment, compression sheathing 72 has a thickness t of 0.001″ (˜0.0254 mm), but can be in the range of about 0.0005″ (˜0.127 mm) to about 0.002″ (˜0.0508 mm). Compression sheathing 72 comprises a polymer that is non-conducting and has low friction characteristics, such as ETFE or mPTFE, or another fluoro-polymer. Compression sheathing 72 should be non-conducting so that it does not interfere with or diminish the electrical signal carried by coil conductor 34.

Compression sheathing 72 must have low friction characteristics so that compression sheathing 72 can rotate within lumen 42D of lead body 42 during deployment and insertion of the fixation helix (tip electrode 12). Compression sheathing 72 is tightly wrapped around coil conductor 34 and is rigid enough so that it restricts the capacity of filars 68 and 70 to expand when placed under torque. Compression sheathing 72 also bonds to coil conductor 34 during extrusion to prevent filars 68 and 70 from contracting in the radial direction, or expanding longitudinally. Thus, compression sheathing 72 enhances the torque transmitting properties of coil conductor 34.

When the proximal end of coil conductor 34 is placed under torque, the windings have a tendency to expand due to the resistance of the tissue on the fixation helix at the distal end. Unless the torque transmitting capacity of the coil exceeds the resistance caused by the tissue, the coil will expand radially rather than rotate the fixation helix. The torque transmitting capacity of the coil is determined by its rigidity, which is influenced by the diameter of the filars and the number of filars. As stated above, typically five filars have been used to reach the desired torque levels. Coil conductor 34 utilizes only two filars with the addition of compression sheathing 72. Compression sheathing 72 prevents radial expansion of coil conductor 34 and instead redirects the energy of the applied rotational force to rotation of coil conductor 34 and its distal end, thereby allowing the fixation helix to penetrate tissue of the heart.

Although the present invention has been described with reference to preferred embodiments, workers skilled in the art will recognize that changes may be made in form and detail without departing from the spirit and scope of the invention.

What is claimed is:

1. A conductor for connecting an electrode near a distal end of a medical electrical lead with an implantable medical device connected with a proximal end of the medical electrical lead, the conductor comprising:
a co-radially wound, multi-filar coil for forming a circuit between the electrode and the implantable medical device, the coil having an RF field compatible inductance; and
a sheathing continuously extending from near a proximal end of the coil to near a distal end of the coil for enhancing torque transmitting properties of the coil.

2. The conductor of claim 1 wherein the electrode comprises a fixation device.

3. The conductor of claim 1 wherein the coil includes a RF field compatible inductance of approximately 1.5 μH or greater.

4. The conductor of claim 1 wherein the co-radially wound, multi-filar coil comprises two filars.

5. The conductor of claim 1 wherein the sheathing comprises a polymer jacket.

6. The conductor of claim 1 wherein the sheathing includes a thickness of approximately 0.001″.

7. The conductor of claim 1 wherein the sheathing is extruded over the multi-filar coil to form a rigid jacket bonded to the coil.

8. A lead for a medical electrical device, the lead comprising:
a lead body including a lumen extending from a proximal end to a distal end;
a co-radially wound, multi-filar coil conductor extending through the lumen and having an RF field compatible inductance; and
a jacket for restricting radial expansion of the multi-filar coil conductor.

9. The lead of claim 8 wherein the co-radially wound, multi-filar coil conductor comprises two filars.

10. The lead of claim 8 wherein the coil conductor includes a RF field compatible inductance of approximately 1.5 μH or greater.

11. The lead of claim 8 wherein the jacket comprises a polymer sheathing.

12. The lead of claim 8 wherein the jacket includes a thickness of approximately 0.001″.

13. The lead of claim 8 wherein the jacket enhances the torque transmitting properties of the multi-filar coil.

14. The lead of claim 8 wherein the coil conductor is connected with a fixation device.

15. The lead of claim 14 wherein the jacket enables the coil conductor to transmit torque for rotating the fixation device.

16. A medical electrical lead comprising:
a lead body;
an electrode fixation device carried at a distal end of the lead body;
a torque coil extending through the lead body from a proximal end to the distal end, the torque coil having winding characteristics such that the inductance of the coil is approximately 1.5 μH or greater; and
a sheathing enveloping the torque coil which enables the torque coil to transmit torque from a proximal end to a distal of the coil end for rotating the fixation device.

17. The lead of claim 16 wherein the torque coil comprises a co-radially wound, bi-filar coil.

18. The lead of claim 16 wherein the sheathing comprises a polymer jacket.

19. The lead of claim 16 wherein the sheathing is extruded over the torque coil to form a rigid jacket bonded to the coil.

20. The lead of claim 16 wherein the sheathing includes a thickness of approximately 0.001″.
